(19) United States
(12) Patent Application Publication
(10) Pub. No.: US 2015/0094611 A1
(43) Pub. Date: Apr. 2, 2015

(54) Method and Device for Improved Hygiene During using Endoscopic accessory tools

(71) Applicant: Ashkan Farhadi, Irvine, CA (US)

(72) Inventor: Ashkan Farhadi, Irvine, CA (US)

(21) Appl. No.: 14/215,005

(22) Filed: Mar. 16, 2014

Related U.S. Application Data
(60) Provisional application No. 61/885,500, filed on Oct. 2, 2013.

(51) Int. Cl. A61B 1/00; A61B 10/04

(52) U.S. Cl. CPC A61B 1/00142; A61B 10/04
USPC 600/562; 600/123

(57) ABSTRACT

An elongated flexible sleeve over the endoscope accessory shaft and secured to the endoscope accessory shaft at the proximal portion protects the shaft of the endoscope accessory from touching the external objects before the endoscope accessory shaft is inserted into endoscope biopsy port and after the endoscope accessory shaft is withdrawn from the endoscope biopsy port. In use, the endoscope accessory cover stays outside of the endoscope biopsy port at all times. The sleeve may be supplied with a handle with a coupling piece at the distal end for removably connect to the endoscope biopsy port cap. The distal end portion of the endoscope accessory cover also may be supplied with an absorbent liner to wipe off secretions during endoscope accessory withdrawal.

FIELD OF INVENTION

This invention relates generally to methods and devices that provide for improved hygiene in the endoscopic examination of body organs, particularly the gastrointestinal tract. The invention relates to providing a cover for an endoscope accessory during an endoscopic exam and is configured to keep the endoscope accessories clean prior to insertion into the patient's body, and to retain any bodily matter within upon removal of the endoscope accessory from the patient's body.

### BACKGROUND OF INVENTION

An endoscope is a well-known optical system for evaluation of internal organs that was disclosed and claimed in U.S. Pat. No. 3,449,037 to C. J. Koester. Currently used fiberoptic endoscopes comprise a flexible tube to relay an image from inside a body cavity for viewing by a physician for diagnosis or manipulation inside cavitary spaces. Most endoscopic procedures are not sterile procedures. During the procedure an endoscope accessory shaft is usually grasped by the endoscopist for insertion, withdrawal, and manipulation. Endoscopes typically contain an accessory channel that runs all along the endoscope accessory shaft and is designed for passing endoscope accessories into a patient's body cavity. The accessories maybe a flexible catheter, or wire like device such as a biopsy forceps that can be removably inserted through the endoscope accessory channels. The endoscope accessory channel is covered by an elastic cap that is usually perforated and traversed by the endoscope accessory. The endoscope accessory shaft is grasped by the endoscopist and its tip is pushed through the endoscope accessory channel cap into the endoscope accessory channel. The accessory can be passed into a body cavity by pushing the endoscope accessory shaft, and removed by pulling the endoscope accessory shaft. After insertion of the endoscope accessory into the endoscope accessory channel, and exposure of the tip of the endoscope accessory into the body cavity, the endoscope accessory shaft will be contaminated with bodily matter. Handling of the endoscope accessory by the endoscopist and endoscopy technician will result in contamination of their hands. After the first use, the endoscope accessory will be handled as a contaminated object, and at the end of the procedure usually will be disposed into special containers. To avoid further contamination of the endoscopist's hands and the endoscopy technician's hands and to improve the sanitization of the endoscopic procedures, there are several policies and techniques in place governed by sanitizing agencies.

Darras in 1989 (Medical instrument cover, U.S. Pat. No. 4,886,049) disclosed a medical instrument cover having an elongated, generally tubular sheath of an elastomeric material, open at both ends, configured to fit on an endoscope having a filament in its interior surface to separate the sheath in an elongate direction and two strips to facilitate the removal of the cover from the endoscope by pulling open the sheath along its length. The designed cover is made from “an elastomeric material to ensure a snug and intimate fit of the sheath on the medical instrument” The sheath is inserted with the endoscope into a body cavity and the inventor suggested “it may be desirable to employ a lubricating agent along the outside of the sheath”. The cover has also a “means for removing the sheath from the medical instrument using a filament while in the body or two stripes while outside of the body”.

Another device is disclosed by Chikama et. Al., U.S. Pat. No. 5,159,919. This device is an endoscope accessory cover comprising a soft disposable cylindrical cover which covers the rigid distal end of an endoscope during endoscope insertion to protect the exposure of the endoscope distal end to the body cavity and improve the cleaning and disinfection process of the endoscope. The cover has a transparent window arranged on a closed face formed on its distal rigid end. The rigid cover is tightly fitted; it is difficult to dismount the rigid cover. The primary object of the invention is to protect the endoscope from contamination during use and to permit immediate reuse of the endoscope.

Another device was disclosed by Hickes, U.S. Pat. No. 5,198,894. This is a “Drape for endoscope” and is an endoscope sleeve-like drape secured in a retracted position at the proximal end of an endoscope. The drape is extended to telescope over and envelope the endoscope CCD camera portion such that the resulting outer surface of the drape in its extended position remains sterile. This device is designed to keep the camera at the end of the endoscope sterile but is not designed for covering the endoscope accessory shaft.

Another device was disclosed in U.S. Pat. No. 5,569,161 by Ebling , et al. in 1996 called “Endoscope with sterile sleeve”. This device has a sterile sleeve that accommodates endoscope fiber optic bundle to avoid the need for sterilizing fiber optic prior to use. A window at the distal end of the sleeve allows viewing through the end of sleeve.

Yet another device was disclosed in U.S. Pat. No. 6,293,907 by Axon , et al called “Endoscope accessory cover having protrusions”. This is a removable cover for an endoscope accessory shaft that includes a sleeve of elastic material having a low friction external surface to improve the frictional resistance to forward movement of the endoscope accessory shaft compared to frictional resistance to reverse movement of the endoscope accessory shaft. The friction between the sleeve and shaft is as such that it prevents relative longitudinal movement of the sleeve and endoscope accessory shaft without requiring the sleeve to be gripped. If desired a lubricant can be applied to the exterior of the sleeve. The frictional resistance differs in the forward and reverse directions, which correspond in use to the insertion and withdrawal direction. The sleeve is inserted with the endoscope accessory shaft inside the body and results in selective pulling of the bowel over the endoscope even when withdrawing force is applied to the endoscope accessory shaft, which in turn facilitates the reduction in loops in the bowel by causing the bowel to pull itself over the endoscope.

Another device is disclosed in U.S. Pat. No. 6,852,077 by Ouchi , et al in 2005 entitled “Cover for preventing contamination of an operating portion of an endoscope”. This is a cover for preventing contamination of an operating portion of an endoscope that is not inserted within the body. The cover is formed in a bag-like shape for enveloping the operating portion by forming a hole for passing the endoscope accessory shaft within the cover on one side and another larger opening in a sleeve-like manner for entering a hand to operate the endoscope operating portion (knob). This device does not cover the endoscope accessory shaft at all.

Another device is disclosed in U.S. Pat. No. 8,262,561 invented by Kress in 2012 entitled “Hygiene protection for endoscopes”. This is a cover for endoscope accessory shaft which is closed at its distal end and is transparent for optical information. It can be rolled up over the shaft of an endoscope in a condom manner in the direction of the axis of the endoscope and includes one or more working and vacuum channels extending in a parallel position in relation to the endoscope and terminating in an open manner on the distal end of the cover. The working channels are only connected to the distal end of the cover but vacuum channel creates a sub-atmospheric pressure within the cover, so the cover adheres tightly to the endoscope accessory shaft for insertion into the body. The cover also has a transparent pane or lens at its said distal end that allows the distal end of the endoscope to face this end window for acquiring images within the body cavity. Again this cover enters the body cavity over the endoscope accessory shaft.

### SUMMARY OF THE INVENTION

The endoscope accessory cover of the present invention is intended to cover a variety of endoscope accessories to improve endoscopic procedure sanitization. Exemplary of the medical instruments which are intended to be covered by the current application are endoscope accessories. Those skilled in the art will recognize that the term endoscope refers to an instrument for visualizing the interior of a hollow organ. Specifically, the covering of the present invention is suitable for use on accessories that can be used with sigmoidoscopes, duodenoscopes, upper G.I. fiberscopes, bronchofiberscopes, colonoscopes and the like.

Therefore, it is an object of this invention to provide an extracorporeal endoscope accessory cover comprising an elongated membranous structure with a proximal end, and a distal end, configured to cover the shaft of an endoscope accessory in a loose slidable relationship, for the purpose of insuring cleanliness of the endoscope accessory during use, and to contain bodily matter disposed upon the endoscope accessory, following use.

In accordance with one aspect of this invention is an extracorporeal endoscope accessory cover comprising an elongated membranous structure with a proximal end, and a distal end; with the proximal end comprising a collar configured for slidable engagement or fixation with a proximal portion of an endoscope accessory shaft; and the distal end comprising a handle configured to hold the distal end of the endoscope accessory cover. The handle terminates in a coupling piece configured for removable engagement with the endoscope biopsy port valve cap. The endoscope accessory cover may be configured to extend beyond the distal end of the endoscope accessory's distal end prior to use of the accessory, and is configured to be pulled over the shaft of the endoscope accessory as the accessory distal end is passed into the endoscope biopsy port.

In accordance with another aspect of this invention is an extracorporeal endoscope accessory cover comprising an elongated membranous structure with a proximal end, and a distal end; with the proximal end comprising a collar configured for slidable engagement or fixation with a proximal portion of an endoscope accessory shaft; and the distal end comprising a handle configured to hold the distal end of the endoscope accessory cover that terminates in a coupling piece configured for removable engagement with the endoscope biopsy port valve cap, wherein the coupling piece is made from rubber and configured to removably connect to the endoscope biopsy port valve cap and act as squeegee to clean the bodily matter off of the accessory shaft during accessory withdrawal.

In addition, a method is disclosed here within for improved hygiene in the endoscopic examination of a patient's body comprising the steps of improving the sanitization of an endoscope accessory tool for use, by covering the endoscope accessory shaft with an extracorporeal endoscope accessory cover comprising an elongated membranous structure with a proximal end, and a distal end; with the proximal end comprising a collar configured for slidable engagement or fixation with a proximal portion of an endoscope accessory shaft; and the distal end comprising a handle configured to hold the distal end of the endoscope accessory cover that terminates in a coupling piece configured for removable engagement with the endoscope biopsy port valve cap, whereby the distal end of the cover is configured to extend beyond the distal end of the endoscope accessory tool prior to insertion of the endoscope accessory distal end into the endoscope biopsy port. The endoscope accessory cover is configured to be connected to the endoscope biopsy port cap using the coupling piece and stay outside of the endoscope biopsy port. During insertion of the endoscope accessory tool, after connecting the coupling piece to the endoscope biopsy port cap, the endoscope accessory tool distal tip is passed into the endoscope biopsy port slidably in relation to the endoscope accessory cover. This allows the distal end of the endoscope accessory tool to enter into the endoscope biopsy port and eventually into the body cavity while the distal handle and the membranous cover stay outside of the endoscope biopsy port at all times. During withdrawal of the endoscope accessory tool, the shaft of the endoscope accessory tool is being pulled out of the endoscope biopsy port slidably in relation to the endoscope accessory cover. This allows the endoscope accessory shaft to stay enclosed within the endoscope accessory cover as endoscope accessory shaft is pulled out of the endoscope biopsy port. Then the distal coupling piece is dis-engaged from the endoscope biopsy port valve cap, and the endoscope accessory is transported for retrieval of its samples. The endoscope accessory cover prevents the contact between the endoscope accessory shaft and the operator hands. This make it possible to keep the endoscope accessory clean before and during the use of the endoscope accessory tool, and keep the hand of operator clean from bodily matter after the use of the endoscope accessory tool. This will also reduce the risk of contamination of the work environment of the endoscopist and endoscopic technician.

### BRIEF DESCRIPTION OF FIGURES

FIG. 1 is a schematic illustration showing the endoscope accessory cover installed over an endoscope accesory tool with the distal end of the endoscope accessory tool extending beyond the distal end of the endoscope accessory cover.

FIG. 2 is a schematic illustration showing the distal end of the endoscope accessory cover prior to being connected to the endoscope biopsy port cap through its distal coupling piece.

FIG. 3 is a cross sectional view taken at Section A-A in FIG. 2.

FIG. 4 is a cross sectional view taken at Section B-B in FIG. 2.

FIG. 5 is a cross sectional view taken at Section C-C in FIG. 2.

### DETAILED DESCRIPTION OF THE INVENTION

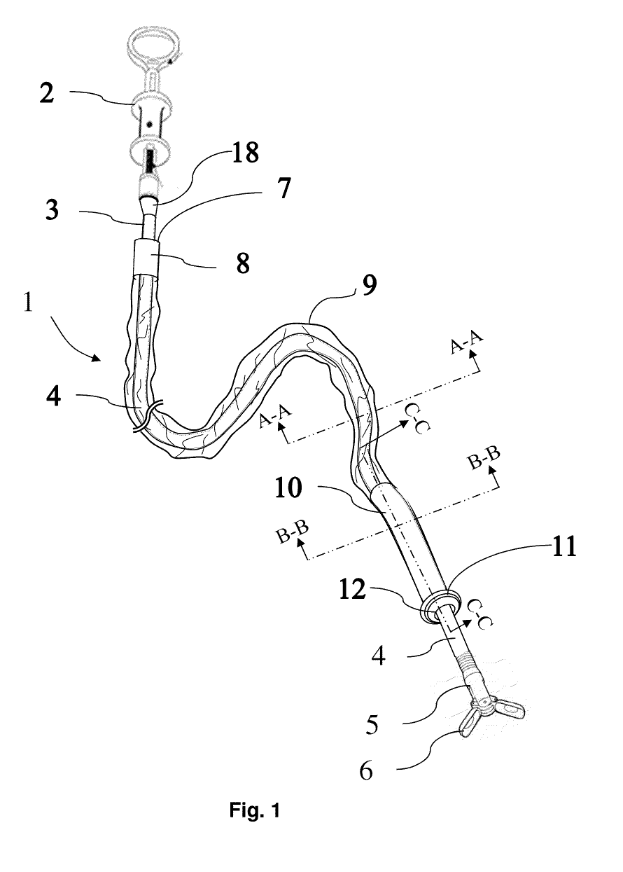

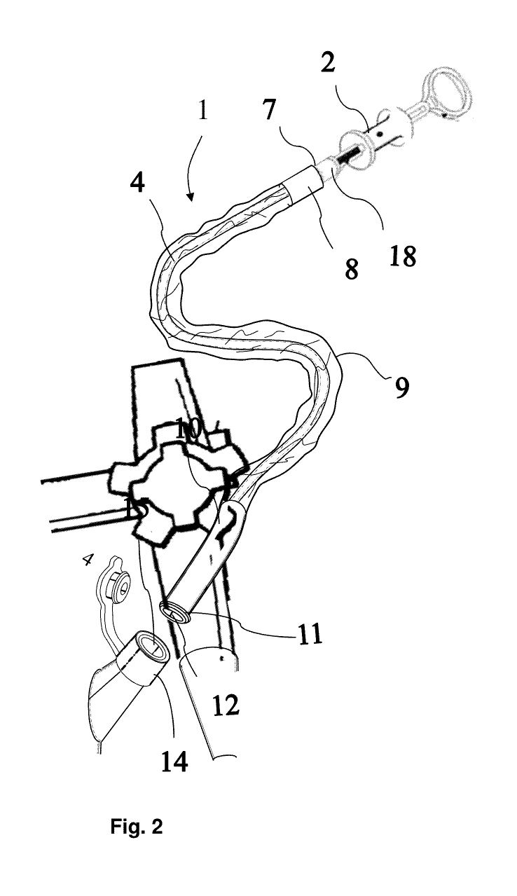

As depicted in FIG. 1 and FIG. 2, endoscope accessory cover 1 comprises endoscope accessory cover sleeve 9, proximal collar 8, disposed on the proximal end of sleeve 9, and handle 10 disposed on the distal end of sleeve 9. Endoscope accessory cover 1 is an elongated flexible sleeve-like cover, having an open proximal end 7 associated with proximal collar 8 and a distal opening 12, associated with handle 10 as shown. Endoscope accessory cover 1 is configured to fit over endoscope accessory shaft 4, defining a passageway for endoscope accessory shaft 4. Sleeve 9 has an interior diameter larger than the exterior diameter of endoscope accessory shaft 4 so that endoscope accessory cover 1 can easily accommodates endoscope accessory shaft 4. Endoscope accessory cover 1 proximal opening 7 can be pulled over distal end portion 5 of endoscope accessory shaft 4 and pulled proximally along the endoscope accessory shaft 4 until proximal opening 7 reaches the proximal portion 3 of endoscope accessory shaft 4 adjacent to endoscope accessory shaft boot 18, which is adjacent to endoscope accessory tool handle 2. Endoscope accessory cover 1 proximal collar 8 can then be secured to the endoscope accessory shaft proximal portion 3 using an interference fit, or an adhesive, which will be described in further detail below. The length of endoscope accessory cover 1 is such that after securing proximal collar 8 to proximal portion 3 of the endoscope accessory shaft 4, distal opening 12 extends beyond the end 6 of endoscope accessory tool as shown in FIG. 2. Handle 10 is disposed at the distal end of sleeve 9 and is distally connected to a coupling piece 11 that can removably engage or disengage with endoscope biopsy port cap 14. Sleeve 9 may comprise an extruded membrane of polymeric material such as polyolefin, with an inner diameter larger than the diameter of endoscope accessory shaft 4. Sleeve 9 may be fabricated from alternative polymeric material providing that the interior surface of sleeve 9 is made from a material that creates minimal friction to the exterior surface of the endoscope accessory shaft 4 and can be reversibly be pleated over the endoscope accessory shaft 4 when the endoscope accessory shaft 4 is passed into the endoscope biopsy port opening 15. Distal coupling piece 11 is the end portion of the handle 10 which is a hollow cylindrical structure and both handle 10 and distal coupling piece 11 are made from an elastomeric material such as silicone rubber, or a urethane rubber with an interior diameter larger than the outer diameter of endoscope accessory shaft 4. Handle 10 may be made from another elastomeric material, or non-elastomeric material.

Just before the insertion of the endoscope accessory tool tip 6 into the endoscope biopsy port opening 15, handle 10 is grasped and the distal coupling piece 11 is engaged to the endoscope biopsy port cap 14 and then the endoscope accessory tool tip 6 is push beyond the distal opening 12 and passes into the endoscope biopsy port opening 15. The endoscope accessory tool distal tip 6 can then be inserted into a body cavity while the proximal portion of the endoscope accessory shaft 4 is enclosed in sleeve 9. Both sleeve 9 and handle 10 with its distal tip 11 stay outside of the endoscope biopsy port opening 15 at all times. During withdrawal of endoscope accessory, shaft 4 is pulled out of endoscope biopsy port 15. When the withdrawal of the endoscope accessory tool from the endoscope biopsy port 15 is complete, handle 10 is pulled so its distal end 11 disengages from the endoscope biopsy port cap 14, while the distal tip of the endoscope accessory tool tip 6 remains within the lumen of the sleeve 9.

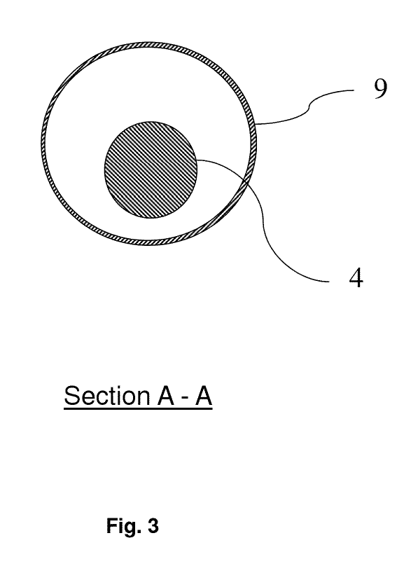

FIG. 3 is a cross sectional view taken at Section A-A of FIG. 1. As shown, sleeve 9 encompasses endoscope accessory shaft 4.

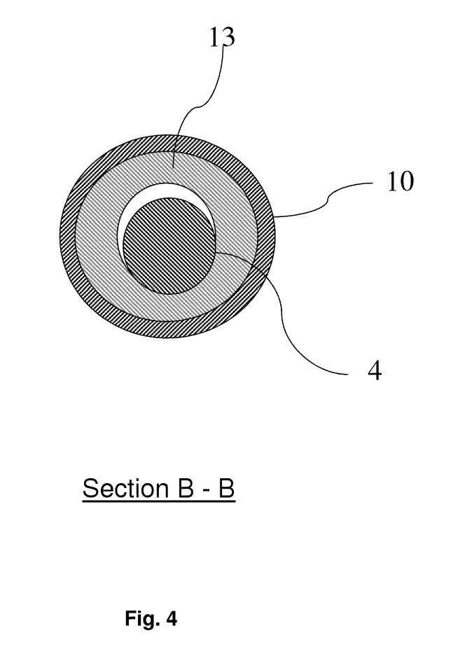

FIG. 4 is a cross sectional view taken at Section B-B of FIG. 1. As shown, handle 10 encompasses endoscope accessory shaft 4. Also it shows an optional embodiment where handle 10 is furnished with an absorbent material 13 disposed on the interior of handle 10. Absorbent material 13 is configured for absorption of bodily matter as endoscope accessory shaft 4 and handle 10 move out of the biopsy port opening 15 in slidable relationship.

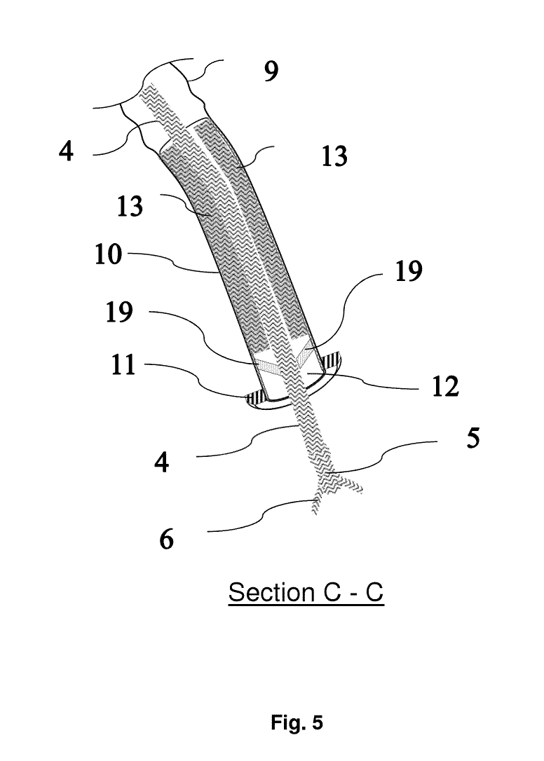

FIG. 5 is a cross sectional view taken at Section C-C of FIG. 1. As shown, handle 10 encompasses endoscope accessory shaft 4. Also it shows an optional embodiment where handle 10 is furnished with an annular squeegee 19 disposed on the interior of handle 10 near its distal end 12. Annular squeegee 19 is a protrusion from the inner surface at an angle of approximately 45 degrees as shown. The inner most diameter of annular squeegee 19 is less than the outer diameter of endoscope accessory shaft 4. The angle, shape, and material properties of annular squeegee 19 provide for a low friction interface between endoscope acccessory shaft 4 and annular squeegee 19, and provides a means for cleaning endoscope acccessory shaft 4 as endoscope acccessory shaft 4 is withdrawn into handle 10 and endoscope cover 9. Optional annular absorbant material 13 may be diposed on the interior of handle 10 as shown, and is configured to absorb residual bodily matter not removed by annular squeegee 19 during withdrawal of endoscope acccessory shaft 4 into endoscope cover 1.

The forgoing description and the drawings are illustrative of the invention and are not to be taken as limiting. Still other variants and rearrangements of structural parts are possible without departing from the spirit and scope of this invention and will readily present themselves to those skilled in the art.

I claim:

1. An endoscope accessory cover comprising:
an elongated flexible sleeve sized to receive an endoscope accessory shaft and having a length sufficient to extend beyond full length of an endoscope accessory shaft, with a proximal end portion, and a distal end portion;
a collar at the proximal end portion sized for removable engagement with the endoscope accessory shaft; and
a handle at the distal end portion configured to connect the distal end of the endoscope accessory cover to the endoscope biopsy port cap.

2. The endoscope accessory cover of claim 1, wherein the collar is elastic and has an inner diameter larger than an insertion portion of the endoscope accessory shaft, but smaller than the diameter of the endoscope accessory shaft at the location of endoscope accessory shaft engagement.

3. The endoscope accessory cover of either claim 1 or 2 wherein the handle is a tube with an inner diameter larger than the endoscope accessory shaft and distally forms a coupling piece that can removably engage and connect to the endoscope biopsy port cap.

4. The endoscope accessory cover of any claims 1 to 3 wherein inner surface of the handle is lined with an absorbent material.

5. The endoscope accessory cover of any claims 1 to 4 wherein an annular squeegee is disposed within the handle.

6. The endoscope accessory cover of claim 5 wherein an annular squeegee is disposed within the handle adjacent to the absorbent material.

7. The endoscope accessory cover of any claims 1 to 6 wherein the elongated, flexible sleeve is a polyolefin tube.

8. A method for improving hygiene during an endoscopic procedure comprising the steps of:
a. introducing an insertable portion of the endoscope accessory shaft into the endoscope accessory cover defined by claim 1; then,
b. connecting the handle coupling piece to the endoscope biopsy port cap; then,
c. inserting the endoscope accessory into the endoscope biopsy port; then,
d. withdrawing of the endoscope accessory tool out of the endoscope biopsy port back into endoscope accessory cover; then,
e. disconnecting the handle coupling piece from the endoscope biopsy port cap; and,
f. sliding the distal end portion of the endoscope accessory cover to expose the distal end of the endoscope accessory to collect the specimen.
